(19) United States
(12) Patent Application Publication
(10) Pub. No.: US 2014/0308229 A1
(43) Pub. Date: Oct. 16, 2014

(54) Aminofunctional Silicone Emulsions For Fiber Treatments

(71) Applicant: Dow Corning Corporation, Midland, MI (US)

(72) Inventors: Sylvie Bouzeloc, Montigny-Le-Tilleul (BE); Severine Cauvin, Mons (BE); Cindy Delvalle, Uccle (BE); Tatiana Dimitrova, Braine-L'Alleud (BE); Sophie Hanssens, Chastre (BE); Benoit Henault, Waterloo (BE); Bethany K. Johnson, Midland, MI (US); Elodie Raynaud, Mons (BE); Houria Seghir, Nivelles (BE); Avril E. Surgenor, Waterloo (BE); Blondine Donatienne Van Roy, Wezembeek-Oppem (BE)

(21) Appl. No.: 14/349,669

(22) PCT Filed: Nov. 28, 2012
(86) PCT No.: PCT/US12/66772

§ 371 Date: Apr. 4, 2014

Related U.S. Application Data
(60) Provisional application No. 61/564,426, filed on Nov. 29, 2011.
(60) Provisional application No. 61/578,483, filed on Dec. 21, 2011.

(51) Int. Cl. A61K 8/898; A61Q 5/10; C11D 3/00; A61Q 5/12

(52) U.S. Cl. CPC A61K 8/898; A61Q 5/12; A61Q 5/10; C11D 3/0015
USPC 424/70.122; 510/527; 8/405

(57) ABSTRACT

Methods for treating fibers are disclosed with aqueous silicone emulsions comprising: A) an aminofunctional organopolysiloxane, B) a quaternary ammonium surfactant having a formula R¹R²R³R⁴N+ X−, where R¹ is an organofunctional group containing at least 10 carbon atoms, R² is R¹ or a hydrocarbyl containing 1 to 12 carbon atoms, R³ is R¹, R₂, or an alcohol group containing 2 to 10 carbon atoms, R⁴ is R¹, R², or R³, X″ is a halide, sulfate, sulfonate, methosulfate, or ethosulfate, C) a nonionic surfactant, where the aqueous silicone emulsion contains less than 0.2 weight % of D4 and D5 cyclic siloxanes.

### CROSS-REFERENCE TO RELATED APPLICATIONS

This application claims the benefit of U.S. Patent Application No. 61/578,483 as filed on Dec. 21, 2011, and U.S. Patent Application No. 61/564426 as filed on Nov. 29, 2011.

### BACKGROUND OF THE INVENTION

Emulsions of aminofunctional silicones are widely used in hair care compositions to provide various aesthetic benefits. Various types of emulsions have been commercially developed to provide water based products of such aminofunctional silicone polymers for use as hair conditioning agents. One method to prepare aminofunctional silicone emulsions involves emulsion polymerization techniques, where siloxane monomers are first emulsified, and then subsequently polymerized to a high molecular weight. Alternatively, mechanical emulsions may be prepared from pre-formed aminofunctional silicones.

Reducing the presence of solvents, un-reacted siloxanes, catalyst residues, cyclic polymerization byproducts, and other impurities in silicone emulsions is an ongoing challenge in the art. The need to reduce such impurities may arise, among other reasons, when such impurities are incompatible with downstream applications (for example, medical, cosmetic, and personal care applications), where the presence of such impurities would reduce the stability of an emulsion, or where regulatory requirements require removal or reduction of their presence. In particular, there is an interest to reduce the presence of cyclosiloxanes, such as octamethylcyclotetrasiloxanes (D4) and decamethylcyclopentasiloxanes (D5), in emulsions of aminofunctional silicones. In many instances D4 and D5 may be present in the process to prepare the aminofunctional silicone emulsions, alternatively they may be produced from side reactions upon storing the emulsion.

### BRIEF SUMMARY OF THE INVENTION

The present inventors have discovered a process for producing mechanical emulsions of aminofunctional siloxanes having reduced content of cyclosiloxanes. Thus, the amount of octamethylcyclotetrasiloxanes (D4) and decamethylcyclopentasiloxanes (D5) in the emulsions produced by the present inventive process is significantly reduced when compared to emulsions prepared by conventional methods. Furthermore, the low D4 and D5 content of the present emulsions remains low with time. In other words, upon shelf aging of the present emulsions, the D4 and D5 content does not significantly increase. The resulting emulsions are particularly useful for treating fibers such as hair or textiles.

The present disclosure relates to methods of treating fibers with aqueous silicone emulsions comprising:
A) an aminofunctional organopolysiloxane,
B) a quaternary ammonium surfactant having a formula

R¹R²R³R⁴N⁺X⁻,
where
   R¹ is an organofunctional group containing at least 10 carbon atoms,
   R² is R¹ or a hydrocarbyl containing 1 to 12 carbon atoms,
   R³ is R¹, R², or an alcohol group containing 2 to 10 carbon atoms,
   R⁴ is R¹, R², or R³,
   X⁻ is a halide, sulfate, sulfonate, methosulfate, or ethosulfate,
C) a nonionic surfactant,
   where the aqueous silicone emulsion contains less than 0.2 weight % of D4 and D5 cyclic siloxanes, and upon ageing the emulsion for one month at 50° C. the content of D4, D5 or both is lower than one of the following:
0.11 wt. % for D4 or 0.12 wt. % for D5 for the emulsion,
below 0.14 for D4 or 0.07 for D5, when the content is expressed as ratio of the cyclic to the non-water content of the cationic surfactant,
below 1.3 for D4 when the content of the later is expressed as ((D4_{AGED}−D4₍ₜ₌₀₎)/ % CS)*100, where D4 is wt % the percentage of D4 in the aged and starting emulsion respectively and % CS is the mass fraction of the cationic surfactant (non-water content) in the emulsion.

### DETAILED DESCRIPTION OF THE INVENTION

A) The Amino functional Organopolysiloxane

Organopolysiloxanes are polymers containing siloxane units independently selected from (R₃SiO_{1/2}), (R₂SiO_{2/2}), (RSiO_{3/2}), or (SiO_{4/2}) siloxy units, where R may be any monovalent organic group. The siloxy units (R₃SiO_{1/2}), (R₂SiO_{2/2}), (RSiO_{3/2}), or (SiO_{4/2}) siloxy units in an organopolysiloxane, are commonly referred to as M, D, T, and Q units respectively. These siloxy units can be combined in various manners to form cyclic, linear, or branched structures. The chemical and physical properties of the resulting polymeric structures can vary. For example organopolysiloxanes can be volatile or low viscosity fluids, high viscosity fluids/gums, elastomers or rubbers, and resins depending on the number and type of siloxy units in the average polymeric formula. R may be any monovalent organic group, alternatively R is a hydrocarbon group containing 1 to 30 carbons, alternatively R is an alkyl group containing 1 to 30 carbon atoms, or alternatively R is methyl.

The organopolysiloxanes useful in the present invention are characterized by having at least one of the R groups in the siloxy unit be an amino group. The amino functional group may be present on any siloxy unit having an R substituent, that is, they may be present on any (R₃SiO_{1/2}), (R₂SiO_{2/2}), or (RSiO_{3/2}) unit, and is designated in the formulas herein as R^{N}. The amino-functional organic group R^{N} is illustrated by groups having the formula; —R³NHR⁴, —R³NR₂⁴, or —R³NHR³NHR⁴, wherein each R³ is independently a divalent hydrocarbon group having at least 2 carbon atoms, and R⁴ is hydrogen or an alkyl group. Each R³ is typically an alkylene group having from 2 to 20 carbon atoms. R³ is illustrated by groups such as; —CH₂CH₂—, —CH₂CH₂CH₂—, —CH₂CH(CH₃)—, —CH₂CH₂CH₂CH₂—, —CH₂CH(CH₃)CH₂—, —CH₂CH₂CH₂CH₂CH₂—, —CH₂CH₂CH₂CH₂CH₂CH₂—, —CH₂CH₂CH(CH₂CH₃)CH₂CH₂CH₂—, —CH₂CH₂CH₂CH₂CH₂CH₂CH₂CH₂—, and —CH₂CH₂CH₂CH₂CH₂CH₂CH₂CH₂CH₂CH₂—. The alkyl groups R⁴ are as illustrated above for R. When R⁴ is an alkyl group, it is typically methyl.

Some examples of suitable amino-functional hydrocarbon groups are; —CH₂CH₂NH₂, —CH₂CH₂CH₂NH₂, —CH₂CH(CH₃)NH₂, —CH₂CH₂CH₂CH₂NH₂, —CH₂CH₂CH₂CH₂CH₂NH₂, —CH₂CH₂CH₂CH₂CH₂CH₂NH₂, —CH₂CH₂NHCH₃, —CH₂CH₂CH₂NHCH₃, —CH₂CH(CH₃)CH₂NHCH₃, —CH₂CH₂CH₂CH₂NHCH₃, —CH₂CH₂NHCH₂CH₂NH₂, —CH₂CH₂CH₂NHCH₂CH₂NH₂, —CH₂CH₂CH₂NHCH₂CH₂CH₂NH₂, —CH₂CH₂CH₂CH₂NHCH₂CH₂CH₂CH₂NH₂, —CH₂CH₂NHCH₂CH₂NHCH₃, —CH₂CH₂CH₂NHCH₂CH₂CH₂NHCH₃, —CH₂CH₂CH₂CH₂NHCH₂CH₂CH₂CH₂NHCH₃, and —CH₂CH₂NHCH₂CH₂NHCH₂CH₂CH₂CH₃.

Alternatively, the amino functional group is —CH₂CH(CH₃)CH₂NHCH₂CH₂NH₂ or CH₂CH₂CH₂NHCH₂CH₂NH₂

The aminofunctional organopolysiloxane used as component A) may be selected from aminofunctional diorganopolysiloxanes containing siloxy units of average formula;

[R₂SiO_{2/2}]ₐ[RR^{N}SiO_{2/2}]_{b}

where;
a is 1-1000, alternatively 1 to 500, alternatively 1 to 200,
b is 1-100, alternatively 1 to 50, alternatively 1 to 10,
   R is independently a monovalent organic group,
alternatively R is a hydrocarbon containing 1-30 carbon atoms,
   alternatively R is a monovalent alkyl group containing 1-12 carbons, or
      alternatively R is a methyl group;
         R^{N} is as defined above. The aminofunctional diorganopolysiloxanes may be terminated with silanol, alkoxy, trialkylsiloxy groups, or mixtures thereof.

The aminofunctional organopolysiloxane used as component A) may also be a combination of any of the aforementioned aminofunctional organopolysiloxanes. The aminofunctional organopolysiloxane may also be dissolved in a suitable solvent, such as a lower molecular weight organopolysiloxane or organic solvent. The aminofunctional organopolysiloxane used as component A) may also be a blend or a mixture of one or several of the afore mentioned aminofunctional organopolysiloxanes with a OH-terminated or trimethyl- or tri-methyl/methoxy PDMS of viscosity of at least 350 cSt at 25° C.

Aminofunctional organopolysiloxanes are known in the art, and many are commercially available. Representative commercial aminofunctional organopolysiloxanes include; XIAMETER® OFX-8040 Fluid, XIAMETER® OHX-8600 Fluid, XIAMETER® OHX-8630 Fluid, XIAMETER® OHX-8803 Fluid, DOW CORNING® AP-8087 Fluid, DOW CORNING® 2-8040 Polymer, DOW CORNING® 8566 Polymer, DOW CORNING® 8600 HYDROPHILIC SOFTENER, and DOW CORNING® 8803 Polymer.

### B) The Quaternary Ammonium Surfactant

Component B) in the present silicone emulsions is a quaternary ammonium surfactant having a formula R¹R²R³R⁴N⁺X⁻,

where
R¹ is an organofunctional group containing at least 10 carbon atoms,
R² is R¹ or a hydrocarbyl containing 1 to 12 carbon atoms,
R³ is R¹, R², or an alcohol group containing 2 to 10 carbon atoms,
R⁴ is R¹, R², or R³,
X⁻ is a halide, sulfate, sulfonate, methosulfate, or ethosulfate.

R¹ is an organofunctional group containing at least 10 carbon atoms, alternatively at least 12 carbon atoms, or alternatively at least 16 carbon atoms. Typically, R¹ contains an organofunctional group such as an ester or amide that links a fatty acid based organic moiety into the quaternary ammonium surfactant molecule. Since R¹ contains an organofunctional group, structural options for R¹ do not include aliphatic hydrocarbons such as long chain alkyl group (for example hexadecyl).

R² is R¹ or a hydrocarbyl containing 1 to 12 carbon atoms. Alternatively, R² is an alkyl group containing 1 to 12 carbon atoms, or alternatively 1 to 6 carbon atoms. Alternatively, R² is methyl.

R³ is R¹, R², or an alcohol group containing 2 to 10 carbon atoms. Alternatively, R³ is an alcohol group containing 2 to 8 carbon atoms, or alternatively 2 to 4 carbon atoms.

Alternatively, R³ is —CH₂CH₂OH.

R⁴ is R¹, R², or R³, as described above.

X⁻ is a halide, sulfate, sulfonate, methosulfate, or ethosulfate. Suitable halides include F⁻, Cl⁻, Br⁻, and I⁻. In certain embodiments X⁻ is Cl⁻ or methosulfate.

In one embodiment, R¹ and R⁴ have the formula R⁵C(O)OR⁶-, where R⁵C(O) is derived from a fatty acid and R⁶ is a divalent hydrocarbon group containing 1 to 4 carbon atoms. In a further embodiment, the fatty acid is oleic acid and R⁶ is —CH₂CH₂—.

In another embodiment, R¹ has the formula R⁵C(O)NHR⁶- where R⁵C(O) is derived from a fatty acid and R⁶ is a divalent hydrocarbon group containing 1 to 4 carbon atoms, and R⁴ is methyl. In a further embodiment, the fatty acid is mink oil and R⁶ is —CH₂CH₂CH₂—.

In one embodiment, the quaternary ammonium surfactant has the formula R¹=R²=R⁵C(O)OCH₂CH₂— where R⁵C(O) is derived from oleic acid, R³ is —CH₂CH₂OH, and R⁴ is methyl. Representative, non-limiting commercial examples for quaternary ammonium surfactants having this structure include Tetranyl® CO-40 (Kao Corporation S.A.).

In one embodiment, the quaternary ammonium surfactant has the formula R¹=R⁵C(O)NHCH₂CH₂CH₂— where R⁵C(O) is derived from mink oil, R² is methyl, R³ is —CH₂CH₂OH, and R⁴ is methyl. Representative, non-limiting commercial examples for quaternary ammonium surfactants having this structure include Incroquat® 26 (Croda Inc. Edison, N.J.).

Alternatively the cationic surfactant could be a mixture of two or more species satisfying the description above.

### C) The Nonionic Surfactant

The present emulsions further contain a nonionic surfactant as component C). The nonionic surfactant may be selected from polyoxyethylene based compounds, such as those considered as ethoxylated alcohols. Representative examples of suitable commercially available nonionic surfactants include polyoxyethylene fatty alcohols sold under the tradename BRIJ® by Croda (ICI Surfactants), Wilmington, Del. Some examples are BRIJ® 35 Liquid, an ethoxylated alcohol known as polyoxyethylene (23) lauryl ether, and BRIJ® 30, another ethoxylated alcohol known as polyoxyethylene (4) lauryl ether. Some additional nonionic surfactants include ethoxylated alcohols sold under the trademark TERGITOL® by The Dow Chemical Company, Midland, Mich. Some example are TERGITOL® TMN-6, an ethoxylated alcohol known as ethoxylated trimethylnonanol; and various of the ethoxylated alcohols, i.e., C₁₂-C₁₄ secondary alcohol ethoxylates, sold under the trademarks TERGITOL® 15-S-5, TERGITOL® 15-S-12, TERGITOL® 15-S-15, and TERGITOL® 15-S-40. Lutensol® supplied by BASF in the series of Lutensol XP known as ethoxylated, C10-Guerbet alcohol and Lutensol TO known as ethoxylated, iso-C13 alcohol may also be used.

Surfactants whole hydrophilic moiety is based on saccharide or poly-saccharide can also be employed. Examples of these are Lutensol® GD70 (BASF) Triton BG-10 from The Dow Chemical Company (Midland, Mich.).

When mixtures containing nonionic surfactants are used, one nonionic surfactant may have a low Hydrophile-Lipophile Balance (HLB) and the other nonionic surfactant may have a high HLB, such that the two nonionic surfactants have a combined HLB of 11 -15, alternatively a combined HLB of 12.5-14.5.

The amount of components A), B), C), and water in the emulsion may vary. Typically, the emulsions will contain;
15 to 80 wt. % of A) aminofunctional polyorganosiloxane,
   alternatively 30 to 75% A) aminofunctional polyorganosiloxane,
   or alternatively 47 to 71% A) aminofunctional polyorganosiloxane,
0.5 to 10wt. % of B) quaternary ammonium surfactant,
   alternatively 1.2 to 8 wt. % of B) quaternary ammonium surfactant,
   or alternatively 1.3 to 6.7wt. % of B) quaternary ammonium surfactant,
2 to 8 wt. % of C) nonionic surfactant,
   alternatively 3 to 7 wt. % of B) nonionic surfactant,
   or alternatively 3.5to 5.2 wt. % of B) nonionic surfactant,
      and sufficient amounts of water, or other components, to sum to 100 wt %.

Other additives can also be incorporated in the emulsions of the present disclosure, such as fillers, viscosity modifiers, foam control agents; anti-freeze agents and biocides.

The present emulsions may be prepared by any known methods, or alternatively prepared by the methods as discussed below.

The present disclosure further provides a process for preparing an emulsion by;
I) forming a mixture comprising;
   A) 100 parts by weight of an aminofunctional organopolysiloxane,
   B) 0.1 to 50 parts by weight of an a quaternary ammonium surfactant,
   C) 0.1 to 50 parts by weight of a non-ionic surfactant,
(components A, B, and C, are as described above)
II) admixing a sufficient amount of water to the mixture from step I) to form an emulsion,
III) optionally, further shear mixing the emulsion and/or diluting of the emulsion with the continuous phase.

The surfactants B) and C) may be added either alone or in combination with varying amounts of water in step I. Typically, when a surfactant or surfactant combination is selected, the surfactant is added in step I as a concentrated aqueous dispersion, or alternatively as an aqueous solution.

The amount of each surfactant added in step I should be 0.1 to 50 parts by weight for every 100 parts by weight of the aminofunctional organopolysiloxane used. Alternatively, the amount of each surfactant added in step I may be 1 to 50 parts by weight for every 100 parts by weight of the aminofunctional organopolysiloxane used. Alternatively, the amount of surfactants added in step I may be 2 to 20 parts by weight for every 100 parts by weight of the aminofunctional organopolysiloxane used.

Mixing in step (I) can be accomplished by any method known in the art to effect mixing of high viscosity materials. The mixing may occur either as a batch, semi-continuous, or continuous process. Mixing may occur, for example using, batch mixing equipments with medium/low shear include change-can mixers, double-planetary mixers, conical-screw mixers, ribbon blenders, double-arm or sigma-blade mixers; batch equipments with high-shear and high-speed dispersers include those made by Charles Ross & Sons (NY), Hockmeyer Equipment Corp. (NJ); batch equipments with high shear actions include Banbury-type (CW Brabender Instruments Inc., NJ) and Henschel type (Henschel mixers America, TX); centrifugal force-based, high shear mixing devices as for example Speed Mixer® (Hauschild & Co KG, Germany). Illustrative examples of continuous mixers/compounders include extruders single-screw, twin-screw, and multi-screw extruders, co-rotating extruders, such as those manufactured by Krupp Werner & Pfleiderer Corp (Ramsey, N.J.), and Leistritz (NJ); twin-screw counter-rotating extruders, two-stage extruders, twin-rotor continuous mixers, dynamic or static mixers or combinations of these equipments.

The temperature and pressure at which the mixing of step I occurs is not critical, but generally is conducted at ambient temperature and pressure. Typically, the temperature of the mixture will increase during the mixing process due to the mechanical energy associated when shearing such high viscosity materials.

Step II of the process involves admixing water to the mixture of step I to form an emulsion. Typically 5 to 2000 parts by weight water are mixed for every 100 parts by weight of the step I mixture to form an emulsion. The water is added to the mixture from step I at such a rate, with additional mixing, so as to form an emulsion of the mixture of step I. While this amount of water can vary depending on the selection of the surfactants, generally the amount of water is from 0.1 to 2000 parts per 100 parts by weight of the step I mixture, alternatively from 5 to 500 parts per 100 parts by weight of the step I mixture, or alternatively from 5 to 100 parts per 100 parts by weight of the step I mixture.

The water added to the mixture from step I may be done in incremental portions, whereby each incremental portion comprises less than 30 weight % of the mixture from step I and each incremental portion of water is added successively to the previous after the dispersion of the previous incremental portion of water, wherein sufficient incremental portions of water are added to form an emulsion of the aminofunctional organopolysiloxane.

Mixing in step (II) can be accomplished by any method known in the art to effect mixing of high viscosity materials. The mixing may occur either as a batch, semi-continuous, or continuous process. Any of the mixing methods as described for step (I), may be used to effect mixing in step (II). Alternatively, mixing in step (II) may also occur via those techniques known in the art to provide high shear mixing to effect formation of emulsions. Representative of such high shear mixing techniques include; homongenizers, sonolators, and other similar shear devices.

Optionally, the emulsion formed in step (II) may be further sheared according to step (III) to reduce particle size and/or improve long term storage stability. The shearing may occur by any of the mixing techniques discussed above. In some cases it might be necessary to run one or several of the steps Ito III under lower pressure or vacuum.

The emulsion products of the present disclosure may be an oil/water emulsion, a water/oil emulsion, a multiple phase or triple emulsion.

In one embodiment, the emulsion products of the present disclosure are oil/water emulsions. The present oil/water emulsions may be characterized by average volume particle of the dispersed organosiloxane block copolymer (oil) phase in the continuous aqueous phase. The particle size may be determined by laser diffraction of the emulsion. Suitable laser diffraction techniques are well known in the art. The particle size is obtained from a particle size distribution (PSD). The PSD can be determined on a volume, surface, length basis. The volume particle size is equal to the diameter of the sphere that has the same volume as a given particle. The term Dv represents the average volume particle size of the dispersed particles. Dv 0.5 is the particle size measured in volume corresponding to 50% of the cumulative particle population. In other words if Dv 0.5=10 μm, 50% of the particle have an average volume particle size below 10 μm and 50% of the particle have a volume average particle size above 10 μm. Unless indicated otherwise all average volume particle sizes are calculated using Dv 0.5.

The average volume particle size of the dispersed siloxane particles in the oil/water emulsions may vary between 0.1 μm and 150 μm; or between 0.1 μm and 30 μm; 0020or between 0.2 μm and 5.0 μm.

The present aminofunctional silicone emulsions are characterized as having less than 0.2 weight % of D4 and D5 cyclic siloxanes. Furthermore, the present aminofunctional silicone emulsions may be characterized as maintaining a low level upon aging of the emulsion. The aging of the present emulsions may be evaluated by storing the emulsion for one month at 50° C. and measuring the D4 and D5 content by gas chromatography (GC) techniques. Upon aging for one month at 50° C. the content D4, D5 or both in the present emulsion is lower than one of the following:
0.11 wt. % for D4 or 0.12 wt. % for D5 for the emulsion,
below 0.14 for D4 or 0.07 for D5, when the content is expressed as ratio of the cyclic to the non-water content of the cationic surfactant,
below 1.3 for D4 when the content of the later is expressed as ((D4_{AGED}−D4₍ₜ₌₀₎)/% CS)*100 where D4 is wt % the percentage of D4 in the aged and starting emulsion respectively and % CS is the mass fraction of the cationic surfactant (non-water content) in the emulsion.

The present emulsions are advantageous over similar aminofunctional emulsions prepared by using long chain aliphatic (such as those derived from fatty acids sources like tallow) based quaternary surfactants. Emulsions based on long chain aliphatic hydrocarbyl quaternary surfactants (for example Arquad 16-29) may produce D4 or D5 in their compositions at pHs other than neutral pH. Thus such emulsions require their pH be adjusted so as to avoid the formation of cyclics (D4 or D5) in the emulsion compositions, or subsequent compositions containing the emulsions. As such, these emulsions may not be suitable in many applications or formulations that are not pH neutral.

Compositions comprising the present emulsions may be formulated into personal care products. The personal care compositions of this invention may be in the form of a cream, a gel, a powder, a paste, or a freely pourable liquid. Generally, such compositions can generally be prepared at room temperature if no solid materials at room temperature are presents in the compositions, using simple propeller mixers, Brookfield counter-rotating mixers, or homogenizing mixers. No special equipment or processing conditions are typically required. Depending on the type of form made, the method of preparation will be different, but such methods are well known in the art.

The personal care products may be functional with respect to the portion of the body to which they are applied, cosmetic, therapeutic, or some combination thereof. Conventional examples of such products include, but are not limited to: antiperspirants and deodorants, skin care creams, skin care lotions, moisturizers, facial treatments such as acne or wrinkle removers, personal and facial cleansers, bath oils, perfumes, colognes, sachets, sunscreens, pre-shave and after-shave lotions, shaving soaps, and shaving lathers, hair shampoos, hair conditioners, hair colorants, hair relaxants, hair sprays, mousses, gels, permanents, depilatories, and cuticle coats, make-ups, color cosmetics, foundations, concealers, blushes, lipsticks, eyeliners, mascara, oil removers, color cosmetic removers, and powders, medicament creams, pastes or sprays including antiacne, dental hygienic, antibiotic, healing promotive, nutritive and the like, which may be preventative and/or therapeutic. In general the personal care products may be formulated with a carrier that permits application in any conventional form, including but not limited to liquids, rinses, lotions, creams, pastes, gels, foams, mousses, ointments, sprays, aerosols, soaps, sticks, soft solids, solid gels, and gels. What constitutes a suitable carrier is readily apparent to one of ordinary skill in the art.

The present compositions can be used in a variety of personal, household, and healthcare applications. In particular, the compositions of the present invention may be used in the personal care products as taught in U.S. Pat. Nos. 6,051,216, 5,919,441, 5,981,680; as disclosed in WO 2004/060271 and WO 2004/060101; in sunscreen compositions as taught in WO 2004/060276; in cosmetic compositions also containing film-forming resins, as disclosed in WO 03/105801; in the cosmetic compositions as taught in US Patent Application Publications 2003/0235553, 2003/0072730, 2003/0170188, EP 1,266,647, EP 1,266,648, EP1,266,653, WO 03/105789, WO 2004/000247 and WO 03/106614; as additional agents to those taught in WO 2004/054523; in long wearing cosmetic compositions as taught in US Patent Application Publication 2004/0180032; in transparent or translucent care and/or make up compositions as discussed in WO 2004/054524.

The use of amino-silicones for various conditioning benefits as for example softness, ease of combing, smoothness etc has been well documented in the patent literature. The conditioning benefits could be conferred either via a standalone conditioner composition or via a shampoo. The amino-silicones are incorporated in these compositions under the form of emulsions. Examples of preferred embodiments can be found in patent documents FR2831800, EP1213333 A2, U.S. Pat. No. 5,756,076A, WO1997012594, FR2748203, WO1997046211, WO1997046210, EP0870491, FR2768616, EP2143417, EP1726293, by L'Oreal; US20080089856, US20090226381 by Procter and Gamble; and WO1999029286, WO1999044567, WO1999044565, US20040062740 by Unilever all of which are incorporated herein by reference.

In yet another aspect the present emulsions can be used as part of colorant of fixative compositions and applied as pre-, during- , post-treatment in the process of coloring or perming hair. The purposes could range from color retention and color enhancement to again conditioning of the colored hair fibers. Examples and preferred embodiments can be found in the patent documents EP1312343A2, EP1312348A2, EP1312349A2, EP1312337, EP1312650, EP1312342 A2, EP1312341 A2, WO2007071684, US20080282482 by L'Oreal and EP1543820 by Procter and Gamble, al of which are incorporated herein by reference.

The compositions according to this invention can be used by the standard methods, such as applying them to the human body, e.g. skin or hair, using applicators, brushes, applying by hand, pouring them and/or possibly rubbing or massaging the composition onto or into the body. Removal methods, for example for color cosmetics are also well known standard methods, including washing, wiping, peeling and the like. For use on the skin, the compositions according to the present invention may be used in a conventional manner for example for conditioning the skin. An effective amount of the composition for the purpose is applied to the skin. Such effective amounts generally range from about 1 mg/cm² to about 3 mg/cm². Application to the skin typically includes working the composition into the skin. This method for applying to the skin comprises the steps of contacting the skin with the composition in an effective amount and then rubbing the composition into the skin. These steps can be repeated as many times as desired to achieve the desired benefit.

The use of the compositions according to the invention on hair may use a conventional manner for conditioning hair. An effective amount of the composition for conditioning hair is applied to the hair. Such effective amounts generally range from about 0.5 g to about 50 g, preferably from about 1 g to about 20 g. Application to the hair typically includes working the composition through the hair such that most or all of the hair is contacted with the composition. This method for conditioning the hair comprises the steps of applying an effective amount of the hair care composition to the hair, and then working the composition through the hair. These steps can be repeated as many times as desired to achieve the desired conditioning benefit.

Non-limiting examples of additives which may be formulated into the personal care compositions in addition to the present emulsions include: additional silicones, anti-oxidants, cleansing agents, colorants, additional conditioning agents, deposition agents, electrolytes, emollients and oils, exfoliating agents, foam boosters, fragrances, humectants, occlusive agents, pediculicides, pH control agents, pigments, preservatives, biocides, other solvents, stabilizers, sun-screening agents, suspending agents, tanning agents, other surfactants, thickeners, vitamins, botanicals, fragrances, waxes, rheology-modifying agents, anti-dandruff, anti-acne, anti-carrie and wound healing-promotion agents.

The personal care composition, such as a shampoo or cleanser may contain at least one anionic detersive surfactant. This can be any of the well-known anionic detersive surfactants typically used in shampoo formulations. These anionic detersive surfactants function as cleansing agents and foaming agents in the shampoo compositions of this invention. The anionic detersive surfactants are exemplified by alkali metal sulforicinates, sulfonated glyceryl esters of fatty acids such as sulfonated monoglycerides of coconut oil acids, salts of sulfonated monovalent alcohol esters such as sodium oleylisethianate, amides of amino sulfonic acids such as the sodium salt of oleyl methyl tauride, sulfonated products of fatty acids nitriles such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanol amine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, alkylarylsulfonates having 1 or more alkyl groups of 8 or more carbon atoms, alkylbenzenesulfonic acid alkali metal salts exemplified by hexylbenzenesulfonic acid sodium salt, octylbenzenesulfonic acid sodium salt, decylbenzenesulfonic acid sodium salt, dodecylbenzenesulfonic acid sodium salt, cetylbenzenesulfonic acid sodium salt, and myristylbenzenesulfonic acid sodium salt, sulfuric esters of polyoxyethylene alkyl ether including CH3(CH2)6CH2O(C2H4O)2SO3H, CH3(CH2)7CH2O(C2H4O)3.5SO3H, CH3(CH2)8CH2O(C2H4O)8SO3H, CH3(CH2)19CH2O(C2H4O)4SO3H, and CH3(CH2)10CH2O(C2H4O)6SO3H, sodium salts, potassium salts, and amine salts of alkylnaphthylsulfonic acid. Preferably the detersive surfactant is selected from the group consisting of sodium lauryl sulfate, ammonium lauryl sulfate, triethanolamine lauryl sulfate, sodium lauryl ether sulfate, and ammonium lauryl ether sulfate. The anionic detersive surfactant is present in the shampoo compositions of this invention in an amount from about 5 to 50 wt % and preferably about 5 to 25 wt % based on the total weight of the composition.

The personal care composition may contain at least one cationic deposition aid, preferably a cationic deposition polymer. The cationic deposition aid will generally be present at levels of from 0.001 to 5%, preferably from about 0.01 to 1%, more preferably from about 0.02% to about 0.5% by weight. The polymer may be a homopolymer or be formed from two or more types of monomers. The molecular weight of the polymer will generally be between 5 000 and 10 000 000, typically at least 10 000 and preferably in the range 100 000 to about 2 000 000. The polymers will have cationic nitrogen containing groups such as quaternary ammonium or protonated amino groups, or a mixture thereof. The cationic charge density has been found to need to be at least 0.1 meq/g, preferably above 0.8 or higher. The cationic charge density should not exceed 4 meq/g, it is preferably less than 3 and more preferably less than 2 meq/g. The charge density can be measured using the Kjeldahl method and should be within the above limits at the desired pH of use, which will in general be from about 3 to 9 and preferably between 4 and 8. The cationic nitrogen-containing group will generally be present as a substituent on a fraction of the total monomer units of the cationic deposition polymer. Thus when the polymer is not a homopolymer it can contain spacer noncationic monomer units. Such polymers are described in the CTFA Cosmetic Ingredient Directory, 3rd edition. Suitable cationic deposition aids include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have Cl-C7 alkyl groups, more preferably C1-3 alkyl groups. Other suitable spacers include vinyl esters, vinyl alcohol, maleic anhydride, propylene glycol and ethylene glycol. The cationic amines can be primary, secondary or tertiary amines, depending upon the particular species and the pH of the composition. In general secondary and tertiary amines, especially tertiary, are preferred. Amine substituted vinyl monomers and amines can be polymerized in the amine form and then converted to ammonium by quaternization. Suitable cationic amino and quaternary ammonium monomers include, for example, vinyl compounds substituted with dialkyl aminoalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g., alkyl vinyl imidazolium, and quaternized pyrrolidine, e.g., alkyl vinyl imidazolium, alkyl vinyl pyridinium, alkyl vinyl pyrrolidine salts. The alkyl portions of these monomers are preferably lower alkyls such as the C,-C., alkyls, more preferably C, and C2 alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide. The cationic deposition aids can comprise mixtures of monomer units derived from amine- and/or quaternary ammonium-substituted monomer and/or compatible spacer monomers. Suitable cationic deposition aids include, for example: copolymers of 1-vinyl-2-pyrrolidine and 1-vinyl-3-methylimidazolium salt (e.g., Chloride salt) (referred to in the industry by the Cosmetic, Toiletry, and Fragrance Association, “CTFA”. as Polyquaternium-16) such as those commercially available from BASF Wyandotte Corp. (Parsippany, N.J., USA) under the LUVIQUAT tradename (e.g., LUVIQUAT FC 370); copolymers of 1-vinyl-2-pyrrolidine and dimethylaminoethyl methacrylate (referred to in the industry by CTFA as Polyquaternium-11) such as those commercially from Gar Corporation (Wayne, N.J., USA) under the GAFQUAT tradename (e.g., GAFQUAT 755N); cationic diallyl quaternary ammonium-containing polymer including, for example, dimethyldiallyammonium chloride homopolymer and copolymers of acrylamide and dimethy1diallyammonium chloride, referred to in the industry (CTFA) as Polyquaternium 6 and Polyquaternium 7, respectively; mineral acid salts of aminoalkyl esters of homo-and co-polymers of unsaturated carboxylic acids having from 3 to 5 carbon atoms, as described in U.S. Pat. No. 4,009,256; and cationic polyacrylamides as described in our copending UK Application No. 9403156.4 (WO95/22311). Other cationic deposition aids that can be used include polysaccharide polymers, such as cationic cellulose derivatives and cationic starch derivatives. Cationic polysaccharide polymer materials suitable for use in compositions of the invention include those of the formula: A-O(R—N⁺R¹R²R³X⁻) wherein: A is an anhydroglucose residual group, such as starch or cellulose anhydroglucose residual, R is an alkylene oxyalklene, polyoxyalkylene, or hydroxyalkylene group, or combination thereof, R′, R˜′ and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R′, R2 and R′) preferably being about 20 or less, and X is an anionic counterion, as previously described. Cationic cellulose is available from Amerchol Corp. (Edison, N.J., USA) in their Polymer IR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200. Other cationic deposition aids that can be used include cationic guar gum derivatives, such as guar hydroxypropyltrimonium chloride (Commercially available from Celanese Corp. in their Jaguar trademark series). Other materials include quaternary nitrogen-containing cellulose ethers (e.g., as described in U.S. Pat. No. 3,962,418, incorporated by reference herein), and copolymers of etherified cellulose and starch (e.g., as described in U.S. Pat. 3,958,581, incorporated by reference herein).

The personal care composition may contain a foam boosting agent. A foam booster is an agent which increases the amount of foam available from a system at a constant molar concentration of surfactant, in contrast to a foam stabilizer which delays the collapse of a foam. Foam building is provided by adding to the aqueous media an effective amount of a foam boosting agent. The foam boosting agent is preferably selected from the group consisting of fatty acid alkanolamides and amine oxides. The fatty acid alkanolamides are exemplified by isostearic acid diethanolamide, lauric acid diethanolamide, capric acid diethanolamide, coconut fatty acid diethanolamide, linoleic acid diethanolamide, myristic acid diethanolamide, oleic acid diethanolamide, stearic acid diethanolamide, coconut fatty acid monoethanolamide, oleic acid monoisopropanolamide, and lauric acid monoisopropanolamide. The amine oxides are exemplified by N-cocodimethylamine oxide, N-lauryl dimethylamine oxide, N-myristyl dimethylamine oxide, N-stearyl dimethylamine oxide, N-cocamidopropyl dimethylamine oxide, N-tallowamidopropyl dimethylamine oxide, bis(2-hydroxyethyl) C12-15 alkoxypropylamine oxide. Preferably a foam booster is selected from the group consisting of lauric acid diethanolamide, N-lauryl dimethylamine oxide, coconut acid diethanolamide, myristic acid diethanolamide, and oleic acid diethanolamide. The foam boosting agent is preferably present in the shampoo compositions of this invention in an amount from about 1 to 15 wt % and more preferably about 2 to 10 wt % based on the total weight of the composition. The composition may further comprise a polyalkylene glycol to improve lather performance. Concentration of the polyalkylene glycol in the shampoo composition may range from about 0.01% to about 5%, preferably from about 0.05% to about 3%, and more preferably from about 0.1% to about 2%, by weight of the composition. The optional polyalkylene glycols are characterized by the general formula: H(OCH2CHR)n-OH wherein R is selected from the group consisting of H, methyl, and mixtures thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist. In the above structure, n has an average value of from about 1500 to about 25,000, preferably from about 2500 to about 20,000, and more preferably from about 3500 to about 15,000. Polyethylene glycol polymers useful herein are PEG-2M wherein R equals H and n has an average value of about 2,000 (PEG-2M is also known as Polyox WSR9 N-10, which is available from Union Carbide and as PEG-2,000); PEG-5M wherein R equals H and n has an average value of about 5,000 (PEG-5M is also known as Polyox WSRO N-35 and Polyox WSRS N-80, both available from Union Carbide and as PEG-5,000 and Polyethylene Glycol 300,000); PEG-7M wherein R equals H and n has an average value of about 7,000 (PEG-7M is also known as Polyox WSRO N-750 available from Union Carbide); PEG-9M wherein R equals H and n has an average value of about 9,000 (PEG 9-M is also known as Polyox WSRS N-3333 available from Union Carbide); and PEG14M wherein R equals H and n has an average value of about 14,000 (PEG-14M is also known as Polyox WSRO N-3000 available from Union Carbide). Other useful polymers include the polypropylene glycols and mixed polyethylene/polypropylene glycols.

The personal care composition may contain a suspending agent at concentrations effective for suspending the preferred silicone conditioning agent, or other water-insoluble material, in dispersed form in the shampoo compositions. Such concentrations range from about 0.001% to about 15%, preferably from about 0.01% to about 5.0%, by weight of the shampoo compositions. Suspending agents include crystalline suspending agents which can be categorized as acyl derivatives, long chain amine oxides, and mixtures thereof, concentrations of which range from about 0.1% to about 5.0%, preferably from about 0.5% to about 3.0%, by weight of the shampoo compositions. These suspending agents are described in U.S. Pat. No. 4,741,855, which description is incorporated herein by reference. These preferred suspending agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable suspending agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); glyceryl esters (e.g., glyceryl distearate) and long chain esters of long chain alkanol amides (e.g., stearamide diethanolamide distearate, stearamide monoethanolamide stearate). Long chain acyl derivatives, ethylene glycol esters of long chain carboxylic acids, long chain amine oxides, and alkanol amides of long chain carboxylic acids in addition to the preferred materials listed above may be used as suspending agents. For example, it is contemplated that suspending agents with long chain hydrocarbyls having C8-C22 chains may be used. Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl (C16-C22) dimethyl amine oxides, e.g., stearyl dimethyl amine oxide. Other suitable suspending agents include xanthan gum at concentrations ranging from about 0.3% to about 3%, preferably from about 0.4% to about 1.2%, by weight of the shampoo compositions. The use of xanthan gum as a suspending agent in silicone containing shampoo compositions is described, for example, in U.S. Pat. No. 4,788,006, which description is incorporated herein by reference. Combinations of long chain acyl derivatives and xanthan gum may also be used as a suspending agent in the shampoo compositions. Such combinations are described in U.S. Pat. No. 4,704,272, which description is incorporated herein by reference. Other suitable suspending agents include carboxyvinyl polymers. Preferred among these polymers are the copolymers of acrylic acid crosslinked with polyallylsucrose as described in U.S. Pat. No. 2,798,053, which description is incorporated herein by reference. Examples of these polymers include Carbopol 934, 940, 941, and 956 available from B. F. Goodrich Company. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer. Other suitable suspending agents may be used in the shampoo compositions, including those that can impart a gel-like viscosity to the composition, such as water soluble or colloidally water soluble polymers like cellulose ethers (e.g., methylcellulose, hydroxybutyl methylcellulose, hyroxypropylcellulose, hydroxypropyl methylcellulose, hydroxyethyl ethylcellulose and hydroxyethylcellulose), guar gum, polyvinyl alcohol, polyvinyl pyrrolidone, hydroxypropyl guar gum, starch and starch derivatives, and other thickeners, viscosity modifiers, gelling agents, etc.

The personal care composition may contain one or more water-soluble emollients including, but not limited to, lower molecular weight aliphatic diols such as propylene glycol and butylene glycol; polyols such as glycerine and sorbitol; and polyoxyethylene polymers such as polyethylene glycol 200. The specific type and amount of water soluble emollient(s) employed will vary depending on the desired aesthetic characteristics of the composition, and is readily determined by one skilled in the art.

The personal care composition may contain various oils. The term “oil” as used herein refers to any material which is substantially insoluble in water. When the composition is to be used in a cosmetic or personal care product, the product components must also be cosmetically acceptable or otherwise meet the conditions of the end use product. Suitable oil components include, but are not limited to, natural oils such as coconut oil; hydrocarbons such as mineral oil and hydrogenated polyisobutene; fatty alcohols such as octyldodecanol; esters such as C12 -C15 alkyl benzoate; diesters such as propylene dipelarganate; and triesters, such as glyceryl trioctanoate and silicones especially cyclomethicone and dimethicone and mixtures thereof. The composition of the invention also contains oils, preferably a mixture of low viscosity and high viscosity oils. Suitable low viscosity oils have a viscosity of 5 to 100 mPa·s at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or mixtures of octyldodecanol, acetylated lanolin alcohol, cetyl acetate, isododecanol, polyglyceryl-3-diisostearate, or mixtures thereof. The high viscosity surface oils generally have a viscosity of 200-1,000,000 mPa·s at 25° C., preferably a viscosity of 100,000-250,000 mPa·s. Surface oils include castor oil, lanolin and lanolin derivatives, triisocetyl citrate, sorbitan sesquioleate, C10-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or mixtures thereof. The suggested ratio of low viscosity to high viscosity oils in the oil phase is 1:15 to 15:1, preferably 1:10 to 10:1 respectively. The preferred formulation of the invention comprises 1 to 20% of a mixture of low viscosity and high viscosity surface oils. Mention may be made, among the optional other non-silicone fatty substances, of mineral oils, such as liquid paraffin or liquid petroleum, of animal oils, such as perhydrosqualene or arara oil, or alternatively of vegetable oils, such as sweet almond, calophyllum, palm, castor, avocado, jojaba, olive or cereal germ oil. It is also possible to use esters of lanolic acid, of oleic acid, of lauric acid, of stearic acid or of myristic acid, for example; alcohols, such as oleyl alcohol, linoleyl or linolenyl alcohol, isostearyl alcohol or octyldodecanol; or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

The personal care composition may contain various waxes. The waxes or wax-like materials generally have a melting point range of 35 to 120° C. at atmospheric pressure. Waxes in this category include synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, or mixtures thereof. The preferred formulation of the invention comprises about 10-30% of a mixture of waxes. Mention may be made, among the waxes capable of being used as non-silicone fatty substances, of animal waxes, such as beeswax; vegetable waxes, such as carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis. Mention may be made, among the silicone waxes, of polymethylsiloxane alkyls, alkoxys and/or esters.

Thickening agent may be added to provide a convenient viscosity. For example, viscosities within the range of 500 to 25,000 mm²/s at 25° C. or more alternatively in the range of 3,000 to 7,000 mm²/s are usually suitable. Suitable thickening agents are exemplified by sodium alginate, gum arabic, polyoxyethylene, guar gum, hydroxypropyl guar gum, ethoxylated alcohols, such as laureth-4 or polyethylene glycol 400, cellulose derivatives exemplified by methylcellulose, methylhydroxypropylcellulose, hydroxypropylcellulose, polypropylhydroxyethylcellulose, starch, and starch derivatives exemplified by hydroxyethylamylose and starch amylose, locust bean gum, electrolytes exemplified by sodium chloride and ammonium chloride, and saccharides such as fructose and glucose, and derivatives of saccharides such as PEG-120 methyl glucose diolate or mixtures of 2 or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent, where used is present in the shampoo compositions of this invention in an amount sufficient to provide a viscosity in the final shampoo composition of from 500 to 25,000 mm²/s. Alternatively the thickening agent is present in an amount from about 0.05 to 10 wt % and alternatively 0.05 to 5 wt % based on the total weight of the composition.

Stabilizing agents can be used in the water phase of the compositions. Suitable water phase stabilizing agents can include alone or in combination one or more electrolytes, polyols, alcohols such as ethyl alcohol, and hydrocolloids. Typical electrolytes are alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate. When the stabilizing agent is, or includes, an electrolyte, it amounts to about 0.1 to 5 wt % and more alternatively 0.5 to 3 wt % of the total composition. The hydrocolloids include gums, such as Xantham gum or Veegum and thickening agents, such as carboxymethyl cellulose. Polyols, such as glycerine, glycols, and sorbitols can also be used. Alternative polyols are glycerine, propylene glycol, sorbitol and butylene glycol. If a large amount of a polyol is used, one need not add the electrolyte. However, it is typical to use a combination of an electrolyte, a polyol and an hydrocolloid to stabilize the water phase, e.g. magnesium sulfate, butylene glycol and Xantham gum.

The composition according to the invention can also be under the form of aerosols in combination with propellant gases, such as carbon dioxide, nitrogen, nitrous oxide, volatile hydrocarbons such as butane, isobutane, or propane and chlorinated or fluorinated hydrocarbons such as dichlorodifluoromethane and dichlorotetrafluoroethane or dimethylether.

Silicone compositions other than the present aminofunctional silicone emulsions, may also be included in the personal care compositions. For example, such silicones include; silicone fluids, gums, resins, elastomers; silicone surfactants and emulsifiers such as silicone polyethers.

Alkylmethylsiloxanes may be included in the present compositions. These siloxane polymers generally will have the formula Me₃SiO[Me₂SiO]_{y}[MeRSiO]_{z}SiMe₃, in which R is a hydrocarbon group containing 6-30 carbon atoms, Me represents methyl, and the degree of polymerization (DP), i.e., the sum of y and z is 3-50. Both the volatile and liquid species of alkymethysiloxanes can be used in the composition.

Silicone gums may be included in the present compositions. Polydiorganosiloxane gums are known in the art and are available commercially. They consist of generally insoluble polydiorganosiloxanes having a viscosity in excess of 1,000,000 centistoke (mm²/s) at 25° C., alternatively greater than 5,000,000 centistoke (mm²/s) at 25° C. These silicone gums are typically sold as compositions already dispersed in a suitable solvent to facilitate their handling. Ultra-high viscosity silicones can also be included as optional ingredients. These ultra-high viscosity silicones typically have a kinematic viscosity greater than 5 million centistoke (mm²/s) at 25° C., to about 20 million centistoke (mm²/s) at 25° C. Compositions of this type in the form of suspensions are most preferred, and are described for example in U.S. Pat. No. 6,013,682 (Jan. 11, 2000).

Silicone resins may be included in the present compositions. These resin compositions are generally highly crosslinked polymeric siloxanes. Crosslinking is obtained by incorporating trifunctional and/or tetrafunctional silanes with the monofunctional silane and/or difunctional silane monomers used during manufacture. The degree of crosslinking required to obtain a suitable silicone resin will vary according to the specifics of the silane monomer units incorporated during manufacture of the silicone resin. In general, any silicone having a sufficient level of trifunctional and tetrafunctional siloxane monomer units, and hence possessing sufficient levels of crosslinking to dry down to a rigid or a hard film can be considered to be suitable for use as the silicone resin. Commercially available silicone resins suitable for applications herein are generally supplied in an unhardened form in low viscosity volatile or nonvolatile silicone fluids. The silicone resins should be incorporated into compositions of the invention in their non-hardened forms rather than as hardened resinous structures.

Silicone carbinol fluids may be included in the present compositions. These materials are described in WO 03/101412 A2, and can be commonly described as substituted hydrocarbyl functional siloxane fluids or resins.

Water soluble or water dispersible silicone polyether compositions may be included in the present compositions: These are also known as polyalkylene oxide silicone copolymers, silicone poly(oxyalkylene) copolymers, silicone glycol copolymers, or silicone surfactants. These can be linear rake or graft type materials, or ABA type where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible.

This disclosure further relates to methods for applying to textile fibers the present silicone emulsions or compositions containing the silicone emulsions, either of which are also referred herein as the treatment composition. The amount applied is a “hand improving” effective amount of the treatment composition and is applied to the fiber and/or textile by any convenient method. Hand for purposes of the invention means the softness and smoothness of the fabric. For example, the treatment composition can be applied by padding, dipping, spraying or exhausting. When the treatment composition comprises more than one solution, dispersion, or emulsion; the solutions, dispersions, and emulsions can be applied simultaneously or sequentially to the textiles. After the treatment composition is applied to the fiber and/or fabric, it can be dried by heat.

The fiber/textile treatment composition can be applied to the fiber and/or textile during making the fibers or textiles, or later such as during laundering textiles. In particular, the silicone emulsions are useful additives in a laundry rinse cycle softener formulations. After application, carriers (if any) can be removed from the treatment composition for example by drying the composition at ambient or elevated temperature. The amount of treatment composition applied to the fibers and textiles is typically sufficient to provide 0.001 to 15 weight percent of the composition on the fibers and textiles, based on their dry weight, preferably in an amount of 0.01 to 5 weight percent based on the dry weight of the fiber or textile.

Fibers and textiles that can be treated with the treatment composition include natural fibers such as cotton, silk, linen, and wool; regenerated fibers such as rayon and acetate; synthetic fibers such as polyesters, polyamides, polyacrylonitriles, polyethylenes, and polypropylenes; combinations, and blends thereof. The form of the fibers can include threads, filaments, tows, yarns, woven fabrics, knitted materials, non-woven materials, paper, carpet, and leather. Textiles treated with the present silicone emulsions have an improved feel or hand comparable to conventional treatments.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %. All measurements were conducted at 23° C. unless indicated otherwise.

### Materials

Unless stated otherwise all cationic emulsifiers are commercial trade names, the aminopolymers are Dow Corning made materials, and percentages refer to mass. CxEy states for a nonionic emulsifiers containing a saturated hydrocarbon chain (linear or branched) of X C-atoms and Y polyoxyethylene units. These materials can be obtained from various manufacturers as for example, but not limited to BASF (Lutensol series), Croda (Synperonic, Brij and Renex series), Clariant (Genapol series) etc.

### Emulsions

Unless stated otherwise, the representative emulsions of the present invention were made using the Dental Speed Mixer DAC 400 FV. 25 g of amino-silicone polymer, the surfactants (cationic and nonionic) and some water in were put in a cup and subjected to a shear to produce emulsions via catastrophic phase inversion. Thus produced concentrated emulsions were then diluted to about 50% silicone. When needed, the pH was adjusted by adding small amounts of 16% or 40% NaOH or acid. Without imposing any restriction, the possible acids are acetic acid, sulfuric acid, hydrochloric acid, citric acid.

### Characterization

In all cases the particle size was measured employing Malvern Mastersizer equipped with a Hydro 2000 sampling unit. Light scattered from the diluted emulsion is collected and analyzed using Mie theory.

The content of D4 and D5 of the starting polymer and freshly prepared emulsions was measured employing gas chromatography. The emulsions were aged at 50° C. for one month and the amount of cyclic was determined on the aged samples as well. The accuracy of the measurement is about 5% of the value reported.

### Performance Evaluation of Emulsions:

Some emulsions were formulated in hair care rinse-off conditioners. Caucasian bleached hair tresses were treated with the rinse-off conditioner formulations containing 2% silicone and forces required to drive a comb through a tress of hair were measured using a Dias-Stron MTT-175 (Dia-Stron Limited, UK). The test was run in an environmentally controlled room with a constant temperature of 20° C.±3° C. and fixed relative humidity of 50%±5%. Total combing load was obtained from UvWin software. Statistical analysis was run with the data generated.

Generally the lower the combing force/load the better the performance. Without being bound to any theory, for the consumer the low combing load translates into one or more of the following:
Ease of detangling
Less entangled
Ease of styling
Smooth/soft
Supple
Reduced friction
Easy to manage
   The list above is not exhaustive and is meant solely to illustrate the importance of the measurement value for the practice. A person skill in the art will understand that different adjectives along the lines above can be used to describe hair characterized with a low dry or wet combing load.

### Drying Time

Hair tresses treated with the rinse-off formulations as described above were subject to a sensory test to assess the drying time. A tress is considered dry if the assessor does not feel humidity/wetness when sliding fingers through the tress. This does not imply that all fibers are necessary dry; the test refers to assessment of the consumer of a hair care formulation.

### Protocol:

Dive each tress into water for 30 seconds
Remove excess water
Hang the tress to a support
Let it dry at RT
Evaluate the drying time

### Conditions of the Test:

Temperature: 20.6° C.
Humidity: 53%

For the consumer the short drying time can also be expressed as
Easy to use formulation
Less hassle
   The list above is not exhaustive and is meant solely to illustrate the importance of the measurement value for the practice. A person skill in the art will understand that different adjectives along the lines above can be used to describe hair characterized with a fast drying time.

### Shine (Luster Value)

Some emulsions were formulated in hair care rinse-off conditioners. Caucasian bleached hair tresses were treated with the rinse-off conditioner formulations. Treatment level corresponds to 0.4 g formulated rinse-off/1 g hair. The rinse off formulations contained 2% Silicone. The tresses (treated and untreated; 3 independent tresses per formulation, 3 reading points per tress) were evaluated for shine/luster using commercial Samba equipment from Bossa Nova Technologies. The instrument measures specular reflection (shine) and second reflection (chroma) and the diffuse reflection of light from the hair to determine the luster value.

For the consumer the increase in luster value can also be expressed as:
Brilliance
Liveliness/Vitality
Healthy
   The list above is not exhaustive and is meant solely to illustrate the importance of the measurement value for the practice. A person skill in the art will understand that different adjectives along the lines above can be used to describe hair characterized with increased shine.

### Rinse Cycle Softener

Replicas of some emulsions from examples 1 to 3 were tested for rinse cycle softening benefits. Emulsion EM3-7t contained 4.1% Tetranyl CO40 in order to match the cationic surfactant-to-polymer ratio of the commercial reference. The laundry tests were performed using European style washers Miele W377, loaded with 5 pillow cases and 4 terry towels. A custom designed water supply ensured a water hardness of 0° F. throughout the whole process.

The wash cycle was a standard pre-programmed washing at 40 C and 600 RPM spinning speed. 20 g commercial washing powder (Dash) were used and commercial, silicone-free softener base was used for the rinse cycle treatment. The silicone emulsions were added to the softener base at concentration of 3% silicone. Upon completion of the wash cycle the towels were dried in a climatic room at 20° C. and 50% RH. Reference towels were generated following the same procedure; but using a commercial emulsion which containing 1-4% cyclic silicones.

The softening benefit of the silicone emulsions was evaluated in via a sensory test based on 16 trained panelists. Each panelist was presented two towels—a reference and a treated one without disclosing which is which. Each panelist was allowed to ply, fold, rub etc. the towels in order to determine which towel is softer. Once the panelist identifies the softer, they were asked to rate the treated towel in a scale from 1 to 10 (1=not soft at all and 10=very soft), pointing them that the score of the reference towel is fixed to 5. Each towel is not used for more than 4 panelists.

When 11 panelists identify a towel as softer the result is deemed to be statistically significant.

### EXAMPLE 1

The polymer used in this example was a 5000 cSt, Dimethyl, Methyl Aminoethylaminoisobutyl siloxane, methoxy & hydroxyl terminated, commercially available under the name DowCorning® AP-8087 fluid. Table 1 and 2 summarizes the emulsion composition and the content of cyclics respectively. Comparative examples are marked with CMP. Asterisks in table 2 show the cases where cyclic are generated either during the preparation of the emulsions or during ageing. These examples show that the emulsions of comparative examples (e.g. the ones stabilized by quaternary ammonium halide) require a control of the pH in order to prevent the formation of cyclics. In contrast, the representative emulsions of the present invention do not require any specific adjustment of the pH.

TABLE 1
Composition of Emulsions containing AP -8087

| Emulsion | Cationic | Nonionic | | Cationic | Nonionic | polymer | |
|---|---|---|---|---|---|---|---|
| Example # | surfactant | Surfactant | pH | surfactant (%) | Surf (%) | (%) | Water |
| E1-1 | Tetranyl | C13E12 | NA | 2.53% | 3.825 | 49.880 | Q.S. |
| | CO-40 | | | | | | 100 |
| E1-2 | Incroquat | C13E12 | NA | 3.07% | 4.192 | 48.820 | Q.S |
| | 26 | | | | | | 100 |
| E1-3 | Arquad | C13E12 | NA | 6.77% | 4.221 | 50.030 | Q.S |
| CMP | 16-29 | | | | | | 100 |
| EM1-4 | Arquad | C13E6 | 8.5 | 6.48% | 4.20 | 49.42 | Q.S |
| CMP | 16-29 | | | | | | 100 |
| EM1-5 | Arquad | C13E6 | 7.2 | 6.48% | 4.20 | 49.35 | Q.S |
| CMP | 16-29 | | | | | | 100 |
| EM1-6 | Arquad | C13E6 | 5.5 | 6.38% | 4.14 | 48.65 | Q.S |
| CMP | 16-29 | | | | | | 100 |
| EM1-7 | Incroquat | C13E6 | 8.5 | 3.05% | 4.15 | 49.49 | Q.S |
| | 26 | | | | | | 100 |
| EM1-8 | Incroquat | C13E6 | 7.2 | 3.05% | 4.14 | 49.37 | Q.S |
| | 26 | | | | | | 100 |
| EM1-9 | Incroquat | C13E6 | 5.5 | 3.02% | 4.08 | 48.76 | Q.S |
| | 26 | | | | | | 100 |
| EM1-10 | Tetranyl | C13E6 | 8.5 | 2.40% | 4.20 | 49.35 | Q.S |
| | CO 40 | | | | | | 100 |
| EM1-11 | Tetranyl | C13E6 | 7.2 | 2.40% | 4.21 | 49.48 | Q.S |
| | CO 40 | | | | | | 100 |
| EM1-12 | Tetranyl | C13E6 | 5.5 | 2.38% | 4.16 | 48.92 | Q.S |
| | CO 40 | | | | | | 100 |

TABLE 2
Cyclics content before and after ageing of emulsions of AP-8087

| | | | D4 (%), | |
|---|---|---|---|---|
| Emulsion | D4 (%) t = 0 | D5 (%) t = 0 | AGED | D5 (%), AGED |
| 8087 amino | 0.11 | 0.21 | | |
| polymer- | | | | |
| E1-1 | 0.055 | 0.127 | 0.11 | 0.13 |
| E1-2 | 0.054 | 0.122 | 0.09 | 0.11 |
| E1-3 CMP | 0.053 | 0.119 | 0.22* | 0.12 |
| EM1-4 CMP | 0.06 | 0.11 | 0.68* | 0.16* |
| EM1-5 CMP | 0.06 | 0.1 | 0.08 | 0.11 |
| EM1-6 CMP | 0.16* | 0.13* | 0.13 | 0.14 |
| EM1-7 | 0.05 | 0.09 | 0.08 | 0.11 |
| EM1-8 | 0.05 | 0.09 | 0.06 | 0.11 |
| EM1-9 | 0.05 | 0.1 | 0.08 | 0.11 |
| EM1-10 | 0.05 | 0.09 | 0.11 | 0.12 |
| EM1-11 | 0.05 | 0.1 | 0.1 | 0.12 |
| EM1-12 | 0.05 | 0.1 | 0.09 | 0.12 |

### EXAMPLE 2

Silicone emulsions were also prepared of a hydroxyl/methoxy terminated aminofunctional polysiloxane having an amine content in the range of 0.02 to 0.2% (mol) amine-substituted Si and viscosity in the range of 54000 to 60000 cSt. The aminofunctional polysiloxanes used in this example were prepared from Sn-catalysed co-condensation of a hydroxyl terminated polydimethylsiloxane of initial viscosity of 5000 cSt and aminoethylaminopropyl-tri-methoxy silane. The process is carried out under vacuum. These polymers were mechanically emulsified using a combination of cationic and nonionic surfactant(s). Table 3 and 4 list the composition and the cyclics content.

TABLE 3
Composition of the emulsion of high viscosity amino-polymer. The
pH was adjusted using small amount of CH3COOH or NaOH, samples EM2-3,
EM2-4, EM2-7, EM2-8 contain 0.3% cellulose based thickener.

| Example | | | | | | | | |
|---|---|---|---|---|---|---|---|---|
| Emulsion | Cationic | Nonionic | | Cationic | Nonionic | Nonionic | Polymer, | |
| # | Surfactant | Surfactants | pH | Surf, % | Surf 1, % | Surf 2, % | % | water |
| EM2-1 | Tetranyl CO | C13E6 | 3.6 | 1.35 | 4.95 | | 60 | Q.S. |
| | 40 | | | | | | | 100 |
| EM2-2 | Tetranyl CO | C13E6 and | 3.6 | 1.35 | 3.15 | 1.8 | 60 | Q.S. |
| | 40 | C13E12 | | | | | | 100 |
| EM2-3 | Tetranyl CO | C13E6 | 5.5 | 1.35 | 4.95 | | 60 | Q.S. |
| | 40 | | | | | | | 100 |
| EM2-4 | Tetranyl CO | C13E6 | 3.6 | 1.35 | 4.95 | | 60 | Q.S. |
| | 40 | | | | | | | 100 |
| EM2-5 | Incroquat 26 | C13E6 and | 3.6 | 1.71 | 4.95 | | 60 | Q.S. |
| | | C13E12 | | | | | | 100 |
| EM2-6 | Incroquat 26 | C13E6 | 3.6 | 1.71 | 3.13 | 1.8 | 60 | Q.S. |
| | | | | | | | | 100 |
| EM2-7 | Incroquat 26 | C13E6 | 5.6 | 1.71 | 4.95 | | 60 | Q.S. |
| | | | | | | | | 100 |
| EM2-8 | Incroquat 26 | C13E6 | 3.6 | 1.71 | 4.95 | | 60 | Q.S. |
| | | | | | | | | 100 |
| EM2- | Arquad 16-29 | C13E6 | 3.6 | 3.51 | 4.92 | | 59 | Q.S. |
| 9CMP | | | | | | | | 100 |

TABLE 4
Cyclics content per gram of dry content of the cationic surfactant (Note
that Arquadis 29% solids). The Amounts of D4 and D5 for starting
polymer were 0.11 and 0.09 respectively.

| | Grams D4 | Grams | | |
|---|---|---|---|---|
| | per gram | D5 per | Grams D4 per | Grams D5 per |
| | dry Cationic | gram dry | gram dry | gram dry |
| Example | surf, | Cationic | Cationic surf, | Cationic surf, |
| Emulsion # | t = 0 | surf, t = 0 | Aged | Aged |
| EM2-1 | 0.075 | 0.060 | 0.119 | 0.063 |
| EM2-2 | 0.075 | 0.060 | 0.144 | 0.067 |
| EM2-3 | 0.075 | 0.060 | 0.121 | 0.064 |
| EM2-4 | 0.075 | 0.060 | 0.104 | 0.067 |
| EM2-5 | 0.061 | 0.052 | 0.106 | 0.058 |
| EM2-6 | 0.061 | 0.052 | 0.099 | 0.056 |
| EM2-7 | 0.061 | 0.052 | 0.093 | 0.055 |
| EM2-8 | 0.061 | 0.052 | 0.071 | 0.051 |
| EM2- | 0.12 | 0.10 | 0.18 | 0.11 |
| 9CMP | | | | |

### EXAMPLE 3

The emulsions of this example where prepared in a similar manner as in examples 1 and 2, but in this example the polymer used was prepared via acid-catalyzed-condensation of OH-terminated polysiloxane and aminoethylaminopropyl-methyl-dialcoxy silane in presence of a end blocker. The method of preparation is described in WO 200316380 and yielded a trimethyl terminated PDMS, with an amine content of less than 1% and viscosity of ca. 1000-2000 cSt. The polymer was stripped prior to emulsification and therefore much lower levels of cyclic silicones are detected. However, for compositions outside of the scope of the invention, the relative increase of the cyclic with respect to the cationic surfactant is larger. Table 5 summarizes the composition and table 6 the cyclics' content. The relative increase in D4 and D5 in table 6 is calculated as follows:

Increase=(% D4aged−% D4start)/% CS*100,

where
% D4aged is the measured % of D4 in the aged emulsion (1 month, 50C),
% D4start is the quantity of the D4 in starting emulsion, supposing that in the moment of preparation the only source of D4 is the aminopolymer
% CS is the percentage of cationic surfactant in the formulation (the non-water content)

TABLE 5
Composition of emulsions containing AP2

| | | Nonionic | Polymer | | Nonionic | Cationic | | |
|---|---|---|---|---|---|---|---|---|
| CODE | | Surfactants | AP2 | pH | Surf, % | Surf, % | Polymer | water |
| EM3-1 | Incroquat | C13E6 | AP2 | 5.5 | 4.09 | 1.97 | 49.32 | Q.S. 100 |
| | 26 | | | | | | | |
| EM3-2 | Incroquat | C13E6 | AP2 | 7.6 | 4.12 | 2.00 | 49.76 | Q.S. 100 |
| | 26 | | | | | | | |
| EM3-3 | Incroquat | C13E6 | AP2 | 8.2 | 4.14 | 2.02 | 50.00 | Q.S. 100 |
| | 26 | | | | | | | |
| EM3-4 | Arquad | C13E6 | AP2 | 8 | 4.17 | 6.41 | 49.97 | Q.S. 100 |
| CMP | 16-29 | | | | | | | |
| EM3-5 | Arquad | C13E6 | AP2 | 5.6 | 4.11 | 6.34 | 49.24 | Q.S. 100 |
| CMP | 16-29 | | | | | | | |
| EM3-6 | Arquad | C13E6 | AP2 | 7 | 4.16 | 6.41 | 49.85 | Q.S. 100 |
| CMP | 16-29 | | | | | | | |
| EM3-7 | Tetranyl | C13E12 | AP2A | 7.3 | 4.16 | 2.43 | 49.49 | Q.S. 100 |
| | CO 40 | | | | | | | |
| EM3-8 | Tetranyl | C13E6 | AP2 | 5.5 | 4.11 | 2.38 | 49.53 | Q.S. 100 |
| | CO 40 | | | | | | | |

TABLE 6
D4 and D5 content of emulsions of AP2

| | | | | | increase | increase |
|---|---|---|---|---|---|---|
| | | | | | D4 per | D5 per |
| | polymer | polymer | D4(%) | D5(%) | cationic | cationic |
| CODE | D4, % | D5, % | AGED | AGED | surf % | surf % |
| EM3-1 | 0.00514 | 0.01137 | 0.024 | 0.011 | 1.10 | 0.28 |
| EM3-2 | 0.00514 | 0.01137 | 0.015 | 0.009 | 0.65 | 0.18 |
| EM3-3 | 0.00514 | 0.01137 | 0.014 | 0.006 | 0.59 | 0.00 |
| EM3-4 | 0.00514 | 0.01137 | 0.062 | 0.009 | 3.19 | 0.20 |
| CMP | | | | | | |
| EM3-5 | 0.00514 | 0.01137 | 0.037 | 0.019 | 1.88 | 0.72 |
| CMP | | | | | | |
| EM3-6 | 0.00514 | 0.01137 | 0.018 | 0.011 | 0.81 | 0.29 |
| CMP | | | | | | |
| EM3-7 | 0.0058 | 0.0136 | 0.0225 | 0.042 | 0.81 | 1.45 |
| EM3-8 | 0.0103 | 0.0198 | 0.019 | 0.012 | 0.59 | 0.09 |

### EXAMPLE 4

Replicas of some of the emulsions from examples 1 and 3 have been formulated in rinse off conditioner at 2% silicone and tested in wet and dry combing against a commercial benchmark. Table 7 summarizes the results. Surprisingly, emulsions according to the invention perform better or are equivalent to the commercial reference, which contains 1 to 5% Octamethylcyclotetrasiloxane.

TABLE 7
Combing load (average of 3 tresses)

| | DRY | Wet | DRY std | WET std dev |
|---|---|---|---|---|
| Emulsion | average | average | dev per | per group |
| Code* | (J) | (J) | group (J) | (J) |
| EM2- | 0.07 | 0.17 | 0.02 | 0.03 |
| 9aCMP | | | | |
| EM1-1 | 0.04 | 0.11** | 0.01 | 0.01 |
| EM1-3CMP | 0.08** | 0.24** | 0.02 | 0.08 |
| EM1-2 | 0.07 | 0.17 | 0.01 | 0.03 |
| DC 949 | 0.06 | 0.11 | 0.01 | 0.02 |

*EM2-9aCMP is the same as EM2-9CMP, but the nonionic surfactant was C13E12 instead of C13/E6.
**These values are statistically different (99% confidence) from the corresponding ones for the commercial reference.
DC949 stands for the commercial reference Dow corning ® 949 Cationic Emulsion

### EXAMPLE 5

TABLE 8
Combing load (average of 3 tresses)

| | | | dry std | wet std |
|---|---|---|---|---|
| Emulsion | dry avg | wet avg | dev | dev |
| code* | (J) | (J) | (J) | (J) |
| EM1-11 | 0.08 | 0.131 | 0.02 | 0.017 |
| EM3-7*** | 0.05 | 0.068** | 0.01 | 0.010 |
| EM1-7 | 0.08 | 0.107** | 0.02 | 0.020 |
| EM1-1 | 0.07 | 0.210** | 0.02 | 0.041 |
| EM3-3 | 0.06 | 0.088** | 0.02 | 0.018 |
| DC 949 | 0.07 | 0.144 | 0.01 | 0.043 |
| EM1-1a | 0.06 | 0.137** | 0.01 | 0.040 |
| EM1-1b | 0.05 | 0.122** | 0.01 | 0.026 |

*Emulsions were replicas of the codes used in examples1-3. Emulsions EM1-1a and EM1-1b contain Lutensol XP79 and Alkyl polyglucoside instead of C13E6.
** These values are statistically different (99% confidence) from the ones for the commercial reference.
One anticipates that emulsions according to the invention perform better or are equivalent to the commercial reference, which contains 1 to 5% Octamethylcyclotetrasiloxane (Source: MSDS).
EM3-7*** is the same as EM3-7 but formulated with C13E6 instead of C13E12.

### EXAMPLE 6

| | dry avg | wet avg | dry std dev | wet std |
|---|---|---|---|---|
| Emulsion Code* | (J) | (J) | (J) | dev (J) |
| EM2-6a | 0.10** | 0.57** | 0.03 | 0.13 |
| EM2-5 | 0.09* | 0.60* | 0.02 | 0.21 |
| EM2-2 | 0.09** | 0.75** | 0.02 | 0.19 |
| EM2-9CMP | 0.06 | 0.77 | 0.01 | 0.16 |
| EM2-7 | 0.09** | 0.74 | 0.02 | 0.26 |
| EM2-2a | 0.09** | 0.78 | 0.02 | 0.18 |
| EM2-1 | 0.08 | 0.50** | 0.03 | 0.12 |
| EM2-1 | 0.10** | 0.64** | 0.02 | 0.11 |
| (1m50C) | | | | |
| EM2-5 | 0.07 | 0.64** | 0.01 | 0.19 |
| (1m50C) | | | | |

*Emulsions were the ones used in Example 2. EM2-2a and EM2-6a contain 0.3% cellulose-based thickener. The abbreviation 1m50C stands for “Emulsionaged 1 month at 50 C.”.
**These values are statistically different (99% confidence) from the ones corresponding to the comparative emulsion, which does generate D4 and D5 upon storage.

### EXAMPLE 7

The emulsions according to this invention provide for a much faster drying than the commercial reference. Dow Corning® 949 cationic emulsion which contains 1 to 5% octamethylcyclotetrasiloxane (source: MSDS).

| | Mean drying time (min); |
|---|---|
| Emulsion Code | Average of 3 |
| EM3-7* | 82 |
| EM3-3 | 86 |
| EM1-11 | 91.3 |
| EM1-7 | 90.6 |
| DC 949 | >100 |

EM3-7* is the same as EM3-7 but formulated with C13E6 instead of C13E12.

### EXAMPLE 8

Replicas of emulsions EM3-7* (Example 7) and EM3-2 has been prepared using the same amount of cationic and nonionic surfactants. Additionally 0.25% of cellulose based thickener and 0.9% biocide have been added to each of them in order to improve stability. These materials were termed EM3-7*A and EM3-2A respectively.

The emulsions according to this invention provide for higher shine than the commercial reference. The commercial reference was Dow Corning® 949 cationic emulsion which contains 1 to 5% octamethylcyclotetrasiloxane (source: MSDS).

| | % change in luster |
|---|---|
| Emulsion Code | treated vs. untreated tresses ± STDEV |
| EM3-7*A | 3.80* ± 3.2 |
| EM3-2A | 7.00* ± 6.2 |
| DC 949 | −11.4 ± 4.9 |

*Statistically different from the reference with confidentiality of 95%

### EXAMPLE 9

The emulsions containing Incroquat 26 and an aminopolymer which is not alkoxy terminated can be used as additives in rinse cycle fabric softeners, providing better or comparable benefits as their CTAC-stabilized analogues.

| | Average | Statistical | Nb of |
|---|---|---|---|
| Products | Score | significance | panelists |
| EM3-7t | 5.47 | NO | 6 |
| Commercial ref | 5.00 | | 10 |
| EM3-3 | 6.41 | YES | 13 |
| Commercial ref | 5.00 | | 3 |
| EM2-1 | 3.81 | YES | 4 |
| EM2-9CMP | 5.00 | | 12 |
| EM2-5 | 5.91 | YES | 13 |
| EM2-9CMP | 5.00 | | 3 |
| EM1-11 | 4.25 | YES | 3 |
| Commercial ref | 5.00 | | 13 |
| EM1-7 | 4.06 | YES | 2 |
| Commercial ref | 5.00 | | 14 |
| EM2-5 | 5.19 | NO | 9 |
| EM3-3 | 5.00 | | 7 |
| EM3-1 | 4.63 | NO | 6 |
| EM3-6CMP | 5.00 | | 10 |
| EM1-7 | 4.75 | NO | 7 |
| EM3-1 | 5.00 | | 9 |

1. A method of treating fibers comprising apply to a fiber an aqueous silicone emulsion containing:
A) an aminofunctional organopolysiloxane,
B) a quaternary ammonium surfactant having a formula:
R¹R²R³R⁴N⁺X⁻,
where
R¹ is an organofunctional group containing at least 10 carbon atoms,
R² is R¹ or a hydrocarbyl containing 1 to 12 carbon atoms,
R³ is R¹, R², or an alcohol group containing 2 to 10 carbon atoms,
R⁴ is R¹, R², or R³,
X⁻ is a halide, sulfate, sulfonate, methosulfate, ethosulfate or phosphate,
C) a nonionic surfactant,
where the aqueous silicone emulsion contains less than 0.2 weight % of D4 and D5 cyclic siloxanes, and upon ageing the emulsion for one month at 50° C. the content of D4, D5 or both is lower than one of the following:
0.11 wt. % for D4 or 0.12 wt. % for D5 for the emulsion,
below 0.1 for D4 or 0.07 for D5, when the content is expressed as ratio of the cyclic to the non-water content of the cationic surfactant,
below 1.3 for D4 when the content of the later is expressed as ((D4_{AGED}−D4₍ₜ₌₀₎)/% CS)*100, where D4 is wt % the percentage of D4 in the aged and starting emulsion respectively and % CS is the mass fraction of the cationic surfactant (non-water content) in the emulsion.

2. The method of claim 1 wherein R¹ and R⁴ have the formula:
R⁵C(O )OR⁶-
where R⁵C(O) is derived from a fatty acid and R⁶ is a divalent hydrocarbon group containing 1 to 4 carbon atoms.

3. The method of claim 2 wherein the fatty acid is oleic acid and R⁶ is —CH₂CH₂—.

4. The method of claim 1 wherein R¹ has the formula:
R⁵C(O)NHR⁶-
where R⁵C(O) is derived from a fatty acid and R⁶ is a divalent hydrocarbon group containing 1 to 4 carbon atoms, and R⁴ is methyl.

5. The method of claim 4 wherein the fatty acid is mink oil and R⁶ is —CH₂CH₂CH₂—.

6. The method of claim 1 where R² is methyl.

7. The method of claim 1 where R³ is —CH₂CH₂OH.

8. The method of claim 1 wherein the aminofunctional organopolysiloxane is a diorganopolysiloxane containing siloxy units of average formula:
[R₂SiO_{2/2}]ₐ[RR^{N}SiO_{2/2}]_{b}
Where:
a is 1-1000, b is 1-100,
R is independently a hydrocarbon containing 1-30 carbon atoms,
R^{N} is an aminofunctional group.

9. The method of claim 8 where the aminofunctional organopolysiloxane is silanol, alkoxy, or trialkysiloxy endcapped.

10. The method of claim 9 where the aminofunctional group has the formula —CH₂CH(CH₃)CH₂NHCH₂CH₂NH₂ or —CH₂CH₂CH₂NH₂ or —CH₂CH₂CH₂NHCH₂CH₂NH₂.

11. The method of claim 1 wherein the nonionic surfactant is an ethoxylated alcohol.

12. The method of claim 1 wherein the nonionic surfactant is an alkyl polyglucoside.

13. The method of claim 1 where the fiber is a hair fiber and the silicone emulsion is applied to hair in a personal care composition.

14. The method of claim 13 where the personal care composition is a shampoo, leave-on conditioner, rinse-off conditioner, hairspray, gel, or styling composition.

15. The method of claim 13 where the personal care composition is a rinse-off conditioner.

16. The method of claim 13 wherein the personal care composition is included in a hair-dyeing composition or is used in pre- or post-treatment in a process of hair-dyeing.

17. The method of claim 4, wherein the aminofunctional organopolysiloxane is a non-alkoxy terminated aminofunctional organopolysiloxane and the silicone emulsion is applied to a textile fiber from a treatment composition.

18. The method of claim 17 where the treatment composition is a rinse cycle treatment formulation.

19. The method of claim 2 where R² is methyl.

20. The method of claim 4 where R² is methyl.
